SEQUENCE LISTING (2 sequences)

SEQ ID NO: 1 - protein, 803 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 2 - protein, 799 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAE

(19) United States
(12) Patent Application Publication
(10) Pub. No.: US 2021/0360914 A1
(43) Pub. Date: Nov. 25, 2021

(54) PRODUCTION OF CELL-BASED VACCINES

(71) Applicant: Heat Biologics, Inc., Durham, NC (US)

(72) Inventors: Damien HALLET, Durham, NC (US); Taylor SCHREIBER, Durham, NC (US)

(21) Appl. No.: 16/763,878

(22) PCT Filed: Dec. 3, 2018
(86) PCT No.: PCT/US2018/063582

§ 371 Date: May 13, 2020

Related U.S. Application Data
(60) Provisional application No. 62/594,317, filed on Dec. 4, 2017.

(51) Int. Cl. A01N 1/02; A61K 39/00

(52) U.S. Cl. CPC A01N 1/0221; A61K 2039/5152; A61K 39/001176; A01N 1/0294

(57) ABSTRACT

The present disclosure provides a method for cell preservation, for example, cryopreservation of cells exposed to ionizing radiation.

### CROSS-REFERENCE TO RELATED APPLICATIONS

This application claims priority to and the benefit of U.S. Provisional Patent Application No. 62/594,317, filed on Dec. 4, 2017, the entire contents of which are herein incorporated by reference herein in their entirety.

### FIELD OF THE DISCLOSURE

The disclosure is directed to cell preservation, for example, cryopreservation of cells exposed to ionizing radiation.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: HTB-028PC_SequenceListing_ST25; date recorded: Nov. 28, 2018; file size: 13.6 KB).

### BACKGROUND

Storage of cells in liquid nitrogen remains the most secure method of cell preservation. Cryopreservation of cells exposed to ionizing radiation (IR) has been shown to induce damage to living cells, however, not much is known about cell response to cryopreservation. Current methods require the availability of freshly inactivated cells at regular intervals during cell culture and requires constant access to a radiation source. Irradiation of frozen cells have been shown to improve function, uniformity and extend their functional lifespan. Irradiated cells while frozen do not experience the effects of the radiation until the frozen cells are thawed. Accordingly, to maintain cell viability the process requires an irradiation facility in close proximity to (and tightly integrated with) the cell culture manufacturing facility. This combination is typically uncommon for industrial scale up. Due to this limitation, there exists a need and an improvement for a process that extends cell longevity and functionality.

### SUMMARY

The present disclosure is based on the surprising discovery that irradiation of cancer vaccine cells following cryopreservation retains cell viability and metabolic functionality.

In some aspects, the disclosure provides a method for preserving cells comprising, obtaining freshly harvested cells in a container; contacting the harvested cells with liquid nitrogen; and administering a dosage of ionizing radiation (IR) to the cells.

In some embodiments, the method further comprises, storing the cells in liquid nitrogen.

In some embodiments, the method increases cell viability.

In some embodiments, the method increases cell recovery.

In some embodiments, the cells are irradiated with gamma radiation. In some embodiments, the irradiation of the cell renders the cell replication incompetent. In some embodiments, the cells are non-proliferative when administered with gamma irradiation. In some embodiments, the dose radiation administered is between 1 (Gy), 5 (Gy), 10 (Gy), 20 (Gy), 30 (Gy), 40 (Gy), 50 (Gy), 60 (Gy), 70 (Gy), 80 (Gy), 90 (Gy), 100 (Gy), 110 (Gy) or 120 (Gy), inclusive of all endpoints

In some embodiments, the dose radiation administered is at least 120 (Gy) gamma radiation.

In some embodiments, the cell expresses a modified and secretable vaccine protein. In some embodiments, the modified and secretable vaccine is a heat shock protein is gp96-Ig.

In some embodiments, the cell is a tumor cell, such as, without limitation, a lung or bladder tumor cell. In some embodiments, the tumor cell is Vesigenurtacel-L (HS-110). In some embodiments, the tumor cell is Vesigenurtacel-L (HS-410).

In some aspects, the method provides for producing a cell comprising a vector encoding a modified and secretable vaccine protein with increased cell viability and/or cell recovery. In some embodiments, the cell is expanded in culture.

In some aspects, the invention relates to a method for making a cancer treatment, by obtaining freshly harvested cells in a container, wherein the cells are tumor cells comprising a vector encoding a modified and secretable vaccine protein; contacting the harvested cells with liquid nitrogen; and administering a dosage of ionizing radiation (IR) to the cells at a dose of at least 120 (Gy). In embodiments, the method further comprises storing the cells in liquid nitrogen. In embodiments, the modified and secretable vaccine protein is gp96-Ig. In embodiments, the tumor cell is vesigenurtacel-L (HS-110) or vesigenurtacel-L (HS-410).

### BRIEF DESCRIPTION OF THE DRAWINGS

FIG. 1 is pictorial showing Vesigenurtacel-L (HS-410) drug product manufacturing process and testing (Phase 2 process).

FIG. 2 is a diagram showing the box configuration and dosimeter position in the cryogenic box. For layers B (bottom), M (middle) or T (top), the dose mapping was performed on one single box per layer, as the cooler rotates during the irradiation. Irradiation exposure is therefore equivalent for corresponding vial positions in each of the four boxes in a given layer.

FIG. 3 shows the irradiation dose rate mapping results in Gray per minute. Red cells (ϕ) indicate Low irradiation doses, green cells (⊙) indicate High irradiation doses.

FIG. 4 is histogram showing replication competency of irradiated and non-irradiated HS-410 Vaccine Cells as Assessed by the CellTrace™ Violet Method. CTV profiles of non-irradiated (red-ϕ), and irradiated (dark blue Δ) HS-410 cells on day 0 (dashed) and day 7 (solid) as well as day 7 profiles of spiked samples at the indicated ratios of non-irradiated to irradiated cells.

FIG. 5 shows the irradiation positions and number of vials assessed by CTV assay for replication. Red cells (ϕ) indicate low irradiation levels, green cells (⊙) indicate high irradiation levels. Each number indicates the number of vials assessed at this relative position from the cooler center, in the indicated layer.

FIG. 6 is histogram showing that irradiation renders HS-410 cells replication-incompetent (CTV assay). CellTrace Violet fluorescence on day 0 vs. day 7 (dashed lines vs. filled) in non-irradiated (red) and irradiated (blue) HS-410 cells. Gating is set by adjusting until ˜95% of non-irradiated cells on day 7 fall into the CTV− gate. The solid curve on the left is HS410 Day 7 while the solid curve on the right is HS410 HD Vial 10001 Day 7.

FIG. 7 shows the irradiation positions and number of vials assessed by CFU assay for replication.

FIG. 8 is a bar graph showing a simulated irradiation study. Interior is the left bar and exterior is the right bar.

FIG. 9 is a histogram showing irradiation renders HS-110 cells replication-incompetent. Representative data showing replication status of cells after irradiation. Dashed lines indicate cells at Day 0; filled peaks indicate cells after seven days of culture. Red (℠) indicates a pre-irradiated sample and blue (Δ) indicates a post-irradiated sample. The shaded curve on the left is HS100 Day 7 and the shaded curve on the right is HS110 Irradiated 1.1 Day 7.

FIG. 10 are a series of histograms showing replication competence of HS-110 vaccine cells irradiated following cryopreservation in individual vials.

FIG. 11 is a pictorial depicting the irradiation and freezing method.

FIG. 12A-B are graphs showing cell recovery and viability of Irradiated/Frozen (Irr/Fr) vs. Frozen/Irradiated cells (Fr/Irr).

FIG. 13A-B are graphs showing HLA-A1 positive cell expression Irradiated/Frozen (Irr/Fr) vs. Frozen/Irradiated cells (Fr/Irr). FIG. 13A shows the HLA-A1 percent positive cells and FIG. 13B shows HLA-A1 expression in isotype and anti HLA-A1 conditions.

FIG. 14A-C are a series of line graphs showing GP96-Ig secretion in Irradiated/Frozen (Irr/Fr) vs. Frozen/Irradiated cells (Fr/Irr). FIG. 14A shows GP96-Ig secretion on Day 1. FIG. 14B shows GP96-Ig secretion on Day 3 and FIG. 14C shows GP96-Ig secretion on Day 5.

FIG. 15 is a bar graph showing ³H-Thymidine uptake in non-irradiated, Irradiated/Frozen (Irr/Fr) and Frozen/Irradiated cells (Fr/Irr). In each series, the order of bars left to right is non-irradiated, Irradiated/Frozen (Irr/Fr) and Frozen/Irradiated cells (Fr/Irr).

FIG. 16 are a series of images showing cell monolayers of non-irradiated, Irradiated/Frozen (Irr/Fr) and Frozen/Irradiated cells (Fr/Irr).

### DETAILED DESCRIPTION OF THE DISCLOSURE

### A. Overview

The present disclosure is based on the discovery that surprisingly irradiation of cancer vaccine cells following cryopreservation retains cell viability and metabolic functionality. The present disclosure improves standard methods of cell cryopreservation, simplifying cell culture of target cells and maximizing research efforts, while minimizing the time and expense of cell handling. The present method provides advantages for application in a commercial, general mass production (GMP) setting, for example, in the scale-up for the production of large cell banks and ease of transportation of the cells. Automation and commercial scale-up overcomes potential contamination problems, finite lifespan, passage-related loss of metabolic capacity, quality control and batch variation. From a commercial perspective, the present method provides a positive benefit and will impact applications ranging from conventional cell, tissue and organ transplantation, through transient cell therapies that disrupt or reduce natural disease progression.

Manufacturing protocols for preservation of cells include an irradiation step following cryopreservation. Aliquoted and cryopreserved cancer vaccine cells in vials are irradiated with gamma radiation on dry ice, such irradiation has been shown to damage the cells' replication machinery rendering the cells replication incompetent while allowing them to stay metabolically active for longer periods of time and to produce chaperone-peptide complexes required for immunization.

In some embodiments the present disclosure provides an improved method for maintaining cell viability without requiring an irradiation facility in close proximity to (and tightly integrated with) the cell culture manufacturing facility. In some embodiments, the method provides the feasibility for an industrial scale up and production for irradiation of cryopreserved and/or frozen vaccinated cells.

In some embodiments, the method ensures that irradiation renders the vaccinated cells replication incompetent. In some embodiments, the method ensures that vaccinated cells lose the ability to proliferate after irradiation.

In some embodiments, the cell contains an expression vector comprising a nucleotide sequence that encodes a secretable vaccine protein. In some embodiments, the cell comprises a vector encoding a modified and secretable heat shock protein (i.e., gp96-Ig). In some embodiments, the cell expresses a modified and secretable heat shock protein (i.e., gp96-Ig). In some embodiments, the vectors provided herein contain a nucleotide sequence that encodes a gp96-Ig fusion protein.

### B. Definitions

“Cryopreservation” is a process where organelles, cells, tissues, extracellular matrix, organs or any other biological constructs susceptible to damage caused by unregulated chemical kinetics are preserved by cooling to very low temperatures (typically −80° C. using solid carbon dioxide or −196° C. using liquid nitrogen). At low enough temperatures, any enzymatic or chemical activity which might cause damage to the biological material in question is effectively stopped. Cryopreservation methods seek to reach low temperatures without causing additional damage caused by the formation of ice during freezing.

“Cultured cells” are typically mammalian cells attached to culture substrates and maintained at 37° C. in conventional cell culture medium such as DMEM, F-12, RPMI 1640, or MCDB 153.

“Cultured-irradiated cells” are cells that have been exposed to a dose of gamma radiation while attached to a flask, a dish or a vial rendering the cells mitotically incompetent. In this case, gamma damage to the cells begins immediately and cannot be delayed.

“Differentiation” is the commitment of a lineage or clone of cells to become a specific cell or tissue type. Differentiation is synonymous with a loss of stem cell characteristics.

“Frozen cells” are cultured cells that have been harvested, concentrated, resuspended in cryoprotectant medium and dispensed in vials or ampoules. These are frozen and stored until needed.

“Frozen-irradiated cells” are frozen cells that are exposed to a dose of gamma radiation while in the frozen state rendering the cells mitotically incompetent. Frozen cells may be packed in crushed dry ice, delivered, irradiated, returned to liquid nitrogen for storage and later use or distribution.

“Freezing” is a process of cooling and storing cells at very low temperatures to maintain cell viability. The technique of cooling and storing cells at a very low temperature permits high rates of cell survivability upon thawing. One substance commonly used in freezing cells is liquid nitrogen which has a temperature of about negative 196° C.

“Gamma induced damage” in mammalian cells is caused by the passage of high energy, short wavelength photons, and other subatomic particles which scatter electrons from atoms and molecules through which they pass, producing trails of peroxides, radicals, and other chemically reactive, cytotoxic species.

A “gamma source” is a device allowing exposure of experimental materials, cells or organisms to specific doses of gamma radiation.

A “gray” or “(Gy)” which has units of joules per kilogram (J/kg), is the SI unit of absorbed dose, and is the amount of radiation required to deposit 1 joule of energy in 1 kilogram of any kind of matter.

### I. Manufacturing of Cell Based Vaccines

The invention provides compositions and methods for the production of cell based vaccines that provide advantages over the processes of the prior art.

### A. Cells of Use in the Invention

The invention finds use with a number of different cells types, particularly those of use as cellular vaccines, which are genetically engineered to include a number of components as outlined herein. In one embodiment, the method provides for the use of a cell comprising a composition containing an expression vector that comprises a nucleotide sequence encoding a secretable vaccine. In some embodiments, the cell comprises a composition containing an expression vector that comprises a nucleotide sequence encoding a secretable gp96-Ig fusion protein. Such a cell, in some embodiments, is irradiated. Such a cell, in some embodiments, is live and attenuated. These cells, in various embodiments, express tumor antigens which may be chaperoned by a vaccine protein (e.g., gp96) of the present method.

A nucleic acid encoding a gp96-Ig fusion sequence can be produced using the methods described in U.S. Pat. Nos. 8,685,384, 8,475,785, 8,968,720, 9,238,064, which are incorporated herein by reference in their entireties.

In some embodiments, the gp96-Ig fusion is encoded on a vector, such as a mammalian expression vector. In some embodiments, the gp96-Ig fusion is a secretable gp96-Ig fusion protein which optionally lacks the gp96 KDEL (SEQ ID NO: 2) sequence. An illustrative amino acid sequence encoding the human gp96 gene of Genbank Accession No. CAA33261:

(SEQ ID NO: 1)
MRALWVLGLCCVLLTFGSVRADDEVDVDGTVEEDLGKSREGSRTDDEVVQ
REEEAIQLDGLNASQIRELREKSEKFAFQAEVNRMMKLIINSLYKNKEIF
LRELISNASDALDKIRLISLTDENALSGNEELTVKIKCDKEKNLLHVTDT
GVGMTREELVKNLGTIAKSGTSEFLNKMTEAQEDGQSTSELIGQFGVGFY
SAFLVADKVIVTSKHNNDTQHIWESDSNEFSVIADPRGNTLGRGTTITLV
LKEEASDYLELDTIKNLVKKYSQFINFPIYVWSSKTETVEEPMEEEEAAK
EEKEESDDEAAVEEEEEEKKPKTKKVEKTVWDWELMNDIKPIWQRPSKEV
EEDEYKAFYKSFSKESDDPMAYIHFTAEGEVTFKSILFVPTSAPRGLFDE
YGSKKSDYIKLYVRRVFITDDFHDMMPKYLNFVKGVVDSDDLPLNVSRET
LQQHKLLKVIRKKLVRKTLDMIKKIADDKYNDTFWKEFGTNIKLGVIEDH
SNRTRLAKLLRFQSSHHPTDITSLDQYVERMKEKQDKIYFMAGSSRKEAE
SSPFVERLLKKGYEVIYLTEPVDEYCIQALPEFDGKRFQNVAKEGVKFDE
SEKTKESREAVEKEFEPLLNWMKDKALKDKIEKAVVSQRLTESPCALVAS
QYGWSGNMERIMKAQAYQTGKDISTNYYASQKKTFEINPRHPLIRDMLRR
IKEDEDDKTVLDLAVVLFETATLRSGYLLPDTKAYGDRIERMLRLSLNID
PDAKVEEEPEEEPEETAEDTTEDTEQDEDEEMDVGTDEEEETAKESTAEK
DEL.

In some embodiments, the gp96 portion of a gp96-Ig fusion can contain all or a portion of a wild type gp96 sequence (e.g., the human sequence set forth in SEQ ID NO: 1. For example, a secretable gp96-Ig fusion protein can include the first 799 amino acids of SEQ ID NO: 1, such that it lacks the C-terminal KDEL (SEQ ID NO: 2) sequence. Alternatively, the gp96 portion of the fusion protein can have an amino acid sequence that contains one or more substitutions, deletions, or additions as compared to the first 799 amino acids of the wild type gp96 sequence, such that it has at least 90% (e.g., at least 90%, at least 91%, at least 92%, at least 93%, at least 94%, at least 95%, at least 96%, at least 97%, at least 98%, or at least 99%) sequence identity to the wild type polypeptide. Thus, in some embodiments, the gp96 portion of nucleic acid encoding a gp96-Ig fusion polypeptide can encode an amino acid sequence that differs from the wild type gp96 polypeptide at one or more amino acid positions, such that it contains one or more conservative substitutions, non-conservative substitutions, splice variants, isoforms, homologues from other species, and polymorphisms.

In some embodiments, the Ig tag in the gp96-Ig fusion comprises the Fc region of human IgG1, IgG2, IgG3, IgG4, IgM, IgA, or IgE, or a variant or fragment thereof. In some embodiments, the expression vector comprises DNA. In some embodiments, the expression vector comprises RNA.

In some embodiments, the cell is obtained from normal or affected subjects, including healthy humans, cancer patients, and patients with an infectious disease, private laboratory deposits, public culture collections such as the American Type Culture Collection, or from commercial suppliers. In some embodiments, the cell is a human tumor cell. In some embodiments, the human tumor cell is a cell from an established NSCLC, bladder cancer, melanoma, ovarian cancer, renal cell carcinoma, prostate carcinoma, sarcoma, breast carcinoma, squamous cell carcinoma, head and neck carcinoma, hepatocellular carcinoma, pancreatic carcinoma, or colon carcinoma cell line. In some embodiments, the human tumor cell line is a NSCLC cell line. In some embodiments, the human tumor cell line is a bladder cancer cell line.

In some embodiments, the cells express a modified and secretable heat shock protein (i.e., gp96-Ig). In some embodiments, the cells express a secretable heat shock protein (i.e., gp96-Ig), for example, Viagenpumantucel-L. Viagenpumatucel-L (HS-110) is a proprietary, allogeneic tumor cell vaccine expressing a recombinant secretory form of the heat shock protein gp96 fusion (gp96-Ig) with potential antineoplastic activity. Upon administration of viagenpumatucel-L, irradiated live tumor cells continuously secrete gp96-Ig along with its chaperoned tumor associated antigens (TAAs) into the blood stream, thereby activating antigen presenting cells, natural killer cells and priming potent cytotoxic T lymphocytes (CTLs) to respond against TAAs on the endogenous tumor cells. Furthermore, Viagenpumatucel-L induces long-lived memory T cells that can fight recurring cancer cells. Viagenpumatucel-L is sometimes referred to in the art as “HS-110”.

In some embodiments, the cells harbor an expression vector comprising a nucleotide sequence that encodes a secretable vaccine protein (i.e., gp96-Ig). In some embodiments, the cells harbor an expression vector comprising a nucleotide sequence that encodes a secretable vaccine protein (i.e., gp96-Ig), for example, Vesigenurtacel-L. Vesigenurtacel-L (HS-410), is a proprietary, allogeneic cell-based therapeutic cancer vaccine expressing a recombinant secretory form of the heat shock protein gp96 fusion (gp96-Ig) which functions dually as an antigen delivery vehicle and adjuvant. Upon administration, Vesigenurtacel-L activates CD8+ T cell responses against a variety of bladder tumor antigens and induces memory T cells capable of fighting recurring cancer cells. Viagenpumatucel-L is sometimes referred to in the art as “HS-410”.

### B. Growth of Cells

Cells may be irradiated and suspended in buffered saline containing human serum albumin (HSA). To avoid possible sources of contamination, cells can be cultured in serum-free, defined medium. Cells may be stored in the same medium supplemented with 20% dimethyl sulfoxide as cryopreservative.

### C. Formulation of Cells

As is known in the art, the cells of the invention must be formulated to allow cryofreezing and subsequent handling, including irradiation. The cell formulations can contain buffers to maintain a preferred pH range, salts or other components that present an antigen to an individual in a composition that stimulates an immune response to the antigen. Cells can be suspended in an appropriate physiological solution, e.g., saline or other pharmacologically acceptable solvent or a buffered solution. Buffered solutions known in the art may contain 0.05 mg to 0.15 mg disodium edetate, 8.0 mg to 9.0 mg NaCl, 0.15 mg to 0.25 mg polysorbate, 0.25 mg to 0.30 mg anhydrous citric acid, and 0.45 mg to 0.55 mg sodium citrate per 1 ml of water so as to achieve a pH of about 4.0 to 5.0. Formulations can also contain one or more pharmaceutically acceptable excipients. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, mannitol, sorbitol, and glycerol.

The physiologically acceptable carrier also can contain one or more adjuvants that enhance the immune response to an antigen. Pharmaceutically acceptable carriers include, for example, pharmaceutically acceptable solvents, suspending agents, or any other pharmacologically inert vehicles for delivering vaccines to a subject. Typical pharmaceutically acceptable carriers include, without limitation: water, saline solution, binding agents (e.g., polyvinylpyrrolidone or hydroxypropyl methylcellulose); fillers (e.g., lactose or dextrose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate).

In some embodiments, the buffer is a saline solution. In some embodiments, cells are irradiated and suspended in buffered saline containing 0.5% HSA. In some embodiments the buffer contains a starch (e.g., pentastarch), which is a subgroup of hydroxyethyl starch, with five hydroxyethyl groups out of each 11 hydroxyls, giving it approximately 50% hydroxyethylation.

In general, a cryopreservative medium is used, generally at a 1:1. In some embodiments, the cryopreservation medium comprises, 20×10⁶ cell/mL, 0.5% HSA, 0.007% sodium bicarbonate, 0.567% sodium chloride, 5% DMSO and 6% Pentastarch. Cells are formulated fresh by a 1:1 dilution with cryopreservation medium to yield a final drug concentration of 20×10⁶ viable cells/mL containing 6% Pentastarch, 5% DMSO, 0.5% HSA, 0.007% sodium bicarbonate and 0.567% sodium chloride.

In some embodiments, the cryopreservation medium comprises, 2×10⁶ cell/mL, 0.5% HSA, 0.007% sodium bicarbonate, 0.567% sodium chloride, 5% DMSO and 6% Pentastarch. Cells are diluted fresh in the wash medium (0.5% HSA, 0.007% sodium bicarbonate and 0.9% sodium chloride) to a concentration of 4×10⁶ cells/mL and immediately formulated by a 1:1 dilution with cryopreservation medium.

### D. Aliquoting of Cells

Once grown, the cells are generally aliquoted into single use vials. Cells are manually dispensed into pre-labeled 1.2 mL cryogenic vials. Cryogenic vials are kept on a cold pack while it is dispended in 30 mL increments to control temperature. Approximately 1,000 cryogenic vials (manufacturing scale) are filled in filling racks and be placed into pre-chilled polycarbonate cryogenic boxes until filling completion. In some embodiments, the cell aliquot is from 10⁵ to 10⁷, with 10⁶ preferred.

### E. Freezing of Cells

Once the cells have been aliquoted, they are frozen. Upon filling completion, the cryogenic boxes are frozen in a controlled rate freezer and stored in the vapor phase of liquid nitrogen freezer prior to irradiation. At this stage, vials are removed for product pre-irradiation characterization and release testing.

The cryogenic boxes containing the vaccine vials (81 product vials per cryogenic box, 12 boxes per LN2 container) are shipped for irradiation in a LN2 dry shipper from the manufacturing site to the irradiation facility. Upon reception, the cryogenic boxes are transferred into a Styrofoam cooler container that has been pre-chilled with dry ice (this excursion on dry ice is <1 hour). The 12 product cryogenic boxes are placed in the cooler using three layers of 4 cryogenic boxes per layer, each layer separated by 2.5 inches of dry ice. The cooler is sealed for irradiation. The specifications for the container, the number of storage boxes of vaccine product and their orientation within the container, the number of frozen product vials per storage box, and the amount and location of dry ice in the container have all been identified and written into a standard operating procedures.

The cooler is then irradiated while rotating on a turntable using Cobalt irradiator (Co⁶⁰). In order to obtain an irradiation dose of ˜120 Gray, the vials are irradiated for approximately 8 to 10 minutes, depending on the algorithm describing the available source decay/radiation level available on the date of irradiation.

The actual dose delivered to the product is based on dose rate on the day of irradiation and exposure time (adjusted for source decay as necessary on the day of irradiation). Irradiation by insertion of a single alanine dosimeter per vaccine batch prior to shipment. This internal dosimeter is read independently as a qualitative test, to assure that the radiation process was conducted. At the end of the irradiation process, the cryogenic boxes are placed back in a LN2 dry shipper and shipped back to the manufacturer. Based on expectations for stability of cryopreserved eukaryotic cells lines through short-term excursions on dry ice, these suitability studies were not repeated for the HS-410 product. However, as all release testing (except mycoplasma) is performed on the finished product post-irradiation and post-thaw, the release testing for the Phase 2 product confirms suitability of this dry ice excursion for the HS-410 product on a lot-by-lot basis.

### F. Irradiation of Cells

As discussed herein, the invention relates to the irradiation of cells after freezing.

The irradiation process utilizes a Cobalt irradiator (Co⁶⁰) to render the cells replication-incompetent, yet still viable to produce the gp96-Ig fusion protein. The final product was formulated, filled into single-dose vials, and placed in cryogenic storage in a non-irradiated state. Only after the product was frozen was it then shipped to a separate facility for irradiation (frozen vials were shipped in LN2 dry shipper units, then transferred to a cooler packed with dry ice for the irradiation process itself). The irradiation process development has consisted of multiple steps, which are described below.

### G. Definition of Cooler Packing Configuration for Irradiation

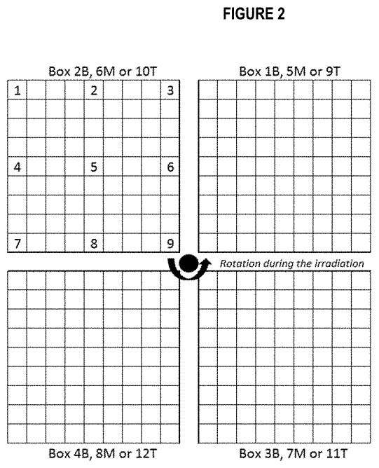

The cooler packing configuration for irradiation is described is shown in FIG. 2. The cooler configuration is a Styrofoam box containing three layers of 4 cryogenic boxes (12 cryogenic boxes in total, each cryogenic box containing 81 vials), each layer separated by 2.5 inches' layer of dry ice. A cooler contains 972 cryogenic vials (batch size). The specifications for the container, the number of storage boxes of vaccine product and their orientation within the container, the number of frozen product vials per storage box, and the amount and location of dry ice in the container have all been identified and written into a standard operating procedure.

### H. Validation of Shipping and Handling Procedures at Irradiation Facility

To ensure that shipping and handling procedures at the irradiation facility did not affect cell viability nor gp96-Ig expression, each of 12 cryogenic boxes contained two frozen vials of non-irradiated HS-110 vaccine (12×10⁶ cells per 0.6 ml per vial) placed in an interior or exterior area of each cryogenic box. The rest of the vial slots of each cryogenic box contained frozen vials of cryopreservative medium. The handling procedures simulated steps for an actual irradiation process and included transfer of the 12 cryogenic boxes from the shipping LN2 dewar to the cooler; storage of the filled cooler at room temperature for 2 hours to simulate a worse case duration for the irradiation process; and transfer from the cooler back into the shipping LN2 dewar. The simulated irradiation was performed at the Steris irradiation facility. After the irradiation simulation, the cryogenic boxes were transferred back into the LN2 shipping dewar and sent back for testing. Twelve vials from the exterior and 12 vials from the interior of the cryogenic boxes were tested for viability and gp96-Ig expression and compared to data generated with cryopreserved cells that were not shipped out for irradiation simulation and kept at the manufacturing site. No difference was observed between cells placed in the interior area of the cryogenic boxes, in the exterior area of the cryogenic boxes or kept at the manufacturing site cryopreserved cells. These data indicate that the shipping and handling procedures to irradiate the cells at a different facility did not adversely affect the vaccine cells.

### I. Storage

Following shipment, the irradiated vials are stored for long term storage in the vapor phase of liquid nitrogen freezer.

### J. Irradiation Dose Mapping within the Irradiation Container

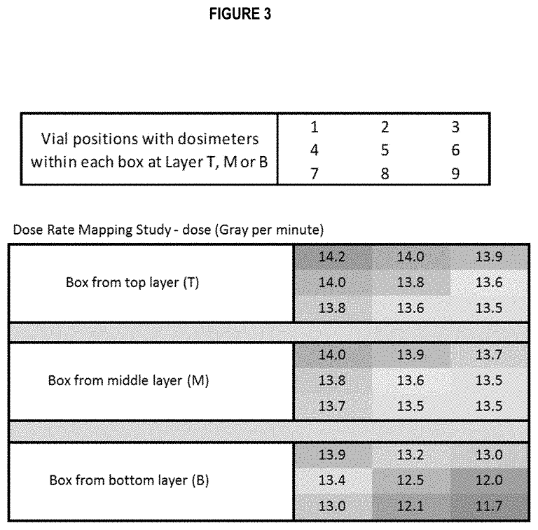

A dose mapping study was conducted to confirm that a dose of ˜120 Gray (Gy) could be delivered to different locations within the 12 cryogenic boxes containing in the Styrofoam cooler. This was performed at room temperature to overcome calibration issues for the dosimeters at sub-zero temperatures. Salt pellets were used to simulate the dry ice (as salt pellets have similar density to dry ice). Dosimeters were at various positions in the cryogenic boxes, and the cooler was irradiated on a turntable. This was repeated three times, and the average irradiation dose for each position was calculated (% RSD ˜2.0%). Based on this configuration, minimum and maximum irradiation dose rates, depending on distance from the source, were calculated to be 11.7 and 14.2 Gray per minute, respectively, for vials at the center of the bottom layer of the cooler (minimum irradiation) and at the exterior corner of the top layer (maximum irradiation). In order to obtain an irradiation dose of ˜120 Gray, the vials should be irradiated for 8.5 to 10.3 minutes, adjusting for source decay as necessary on the day of irradiation. Given the results, the expected range of irradiation received for individual product vials in this process would range from a minimum ˜108 Gray to a maximum ˜132 Gray, (see FIG. 3). Moving forward with cGMP processing, the actual dose delivered to the product was based on dose rate on the day of irradiation and exposure time. For future product batches, irradiation is independently confirmed by via insertion of a single alanine dosimeter per vaccine batch prior to shipment, and also by at least 2 dosimeters placed on opposite corners of the Styrofoam cooler (to confirm appropriate cooler rotation during irradiation). These dosimeters are read at NIST to assure that the irradiation process was conducted, and to assess the irradiation dose received.

### K. Dosing

Many commonly used dose measuring or dosimetry methods are influenced by temperature making the placement of a dosimeter in the volume of frozen material impractical. The use of a reference dosimeter monitoring location, with an empirically determined correction factor, eliminates the need to compensate for the difference in dosimeter response due to temperatures. A simulate material which mimics the density and distribution of the proposed subject material or actual material that will not be distributed to market, at ambient temperature, can be used to establish the dose ratios (and resulting correction factors), thus avoiding temperature compromise to the dosimeter results. Once a ratio has been determined using a representative material at ambient temperature, a routine dosimetry system can be used to measure the reference dose. The minimum and maximum doses can then be calculated by applying the established correction factors to the measured reference dose. When the dose range required to be delivered to a product is below the measurement capabilities of the dosimetry system in use at the time of irradiation, dose rates may be used in place of dosimeters during processing. Both a minimum and maximum dose rate can be determined for the product based on the exposure time, average minimum delivered dose and average maximum dose imparted over three irradiation runs conducted under the same processing conditions and adjusted for decay of the radioactive source. The calculated minimum and maximum dose rates are specific to the turn table and position for which they are calculated. Once calculated, the dose rates can be used to determine the irradiation processing time and dose delivered during irradiation.

In some embodiments, the dose rate is about 0.1 (Gy), 0.2 (Gy), 0.3 (Gy), 0.4 (Gy), 0.5 (Gy), 0.6 (Gy), 0.7 (Gy), 0.8 (Gy), 0.9 (Gy), 1 (Gy), 5 (Gy), 10 (Gy), 15 (Gy), 20 (Gy), 25 (Gy), 30 (Gy), 35 (Gy), 40 (Gy), 45 (Gy), 50 (Gy), 55 (Gy), 60 (Gy), 65 (Gy), 70 (Gy), 75 (Gy), 80 (Gy), 85 (Gy), 90 (Gy), 95 (Gy), 100 (Gy), 110 (Gy), 115 (Gy), 120 (Gy), 125 (Gy), 130 (Gy), 135 (Gy), 140 (Gy), 145 (Gy), 150 (Gy), 155 (Gy), 160 (Gy), 165 (Gy), 170 (Gy), 175 (Gy), 180 (Gy), 185 (Gy), 190 (Gy), 195 (Gy), 200 (Gy), 210 (Gy), 215 (Gy), 220 (Gy), 225 (Gy), 230 (Gy), 235 (Gy), 240 (Gy), 245 (Gy), 250 (Gy), 255 (Gy), 260 (Gy), 265 (Gy), 270 (Gy), 275 (Gy), 280 (Gy), 285 (Gy), 290 (Gy), 295 (Gy), 300 (Gy), 320 (Gy), 325 (Gy), 330 (Gy), 35 (Gy), 340 (Gy), 350 (Gy), 360 (Gy), 365 (Gy), 370 (Gy), 375 (Gy), 380 (Gy), 385 (Gy), 390 (Gy), 400 (Gy), 425 (Gy), 430 (Gy), 435 (Gy), 440 (Gy), 445 (Gy), 450 (Gy), 460 (Gy), 465 (Gy), 470 (Gy), 475 (Gy), 480 (Gy), 485 (Gy), 490 (Gy), 495 (Gy), 500 (Gy), 525 (Gy), 530 (Gy), 535 (Gy), 540 (Gy), 545 (Gy), 550 (Gy), 560 (Gy), 565 (Gy), 570 (Gy), 575 (Gy), 580 (Gy), 585 (Gy), 590 (Gy), 595 (Gy), 600 (Gy), 625 (Gy), 630 (Gy), 635 (Gy), 640 (Gy), 645 (Gy), 650 (Gy), 660 (Gy), 665 (Gy), 670 (Gy), 675 (Gy), 680 (Gy), 685 (Gy), 690 (Gy), 695 (Gy), 700 (Gy), 725 (Gy), 730 (Gy), 735 (Gy), 740 (Gy), 745 (Gy), 750 (Gy), 755 (Gy), 760 (Gy), 765 (Gy), 775 (Gy), 780 (Gy), 785 (Gy), 790 (Gy), 795 (Gy), 800 (Gy), 825 (Gy), 830 (Gy), 835 (Gy), 840 (Gy), 845 (Gy), 850 (Gy), 855 (Gy), 860 (Gy), 865 (Gy), 870 (Gy), 875 (Gy), 880 (Gy), 885 (Gy), 890 (Gy), 900 (Gy), 925 (Gy), 930 (Gy), 935 (Gy), 940 (Gy), 945 (Gy), 950 (Gy), 955 (Gy), 960 (Gy), 965 (Gy), 970 (Gy), 975 (Gy), 980 (Gy), 985 (Gy), 995 (Gy), or 1,000 (Gy), inclusive of the endpoints.

In some embodiments, the dose rate is about 20 (Gy), 25 (Gy), 30 (Gy), 35 (Gy), 40 (Gy), 45 (Gy), 50 (Gy), 55 (Gy), 60 (Gy), 65 (Gy), 70 (Gy), 75 (Gy), 80 (Gy), 85 (Gy), 90 (Gy), 95 (Gy), 100 (Gy), 110 (Gy), 115 (Gy), 120 (Gy), 125 (Gy), 130 (Gy), 135 (Gy), 140 (Gy), 145 (Gy), 150 (Gy), 155 (Gy), 160 (Gy), 165 (Gy), 170 (Gy), 175 (Gy), 180 (Gy), 185 (Gy), 190 (Gy), 195 (Gy), 200 (Gy), 210 (Gy), 215 (Gy), 220 (Gy), 225 (Gy), 230 (Gy), 235 (Gy), 240 (Gy), 245 (Gy), 250 (Gy), 255 (Gy), 260 (Gy), 265 (Gy), 270 (Gy), 275 (Gy), 280 (Gy), 285 (Gy), 290 (Gy), 295 (Gy), 300 (Gy), 320 (Gy), 325 (Gy), 330 (Gy), 35 (Gy), 340 (Gy), 350 (Gy), 360 (Gy), 365 (Gy), 370 (Gy), 375 (Gy), 380 (Gy), 385 (Gy), 390 (Gy), 400 (Gy), 425 (Gy), 430 (Gy), 435 (Gy), 440 (Gy), 445 (Gy), 450 (Gy), 460 (Gy), 465 (Gy), 470 (Gy), 475 (Gy), 480 (Gy), 485 (Gy), 490 (Gy), 495 (Gy), 500 (Gy), inclusive of the endpoints. In some embodiments, the dose rate is 120 (Gy). In some embodiments, aliquot and cryopreserved cancer vaccine cells in vials are irradiated with 120 (Gy) on dry ice.

In some embodiments, the dose rate is about 0.1 (kGy), 0.2 (kGy), 0.3 (kGy), 0.4 (kGy), 0.5 (kGy), 0.6 (kGy), 0.7 (kGy), 0.8 (kGy), 0.9 (kGy), 1 (kGy), 25 (kGy), 50 (kGy), 75 (kGy), 100 (kGy), 125 (kGy), 150 (kGy), 175 (kGy), 200 (kGy), 225 (kGy), 250 (kGy), 275 (kGy), 300 (kGy), 325 (kGy), 350 (kGy), 375 (kGy), 400 (kGy), 425 (kGy), 450 (kGy), 475 (kGy), 500 (kGy), 525 (kGy), 550 (kGy), 575 (kGy), 600 (kGy), 625 (kGy), 650 (kGy), 675 (kGy), 700 (kGy), 725 (kGy), 750 (kGy), 775 (kGy), 800 (kGy), 825 (kGy), 850 (kGy), 875 (kGy), 900 (kGy), 925 (kGy), 950 (kGy), 975 (kGy) or 1,000 (kGy), inclusive of the endpoints.

In some embodiments, the cells are irradiated for about 1 to 2 minutes, 2 to 3 minutes, 3 to 4 minutes, 4 to 5 minutes, 5 to 6 minutes, 6 to 7 minutes, 7 to 8 minutes, 8 to 9 minutes, 9 to 10 minutes, 10 to 11 minutes, 11 to 12 minutes, 12 to 13 minutes, 13 to 14 minutes, 14 to 15 minutes, 15 to 16 minutes, 16 to 17 minutes, 17 to 18 minutes, 18 to 19 minutes, 19 to 20 minutes, 20 to 21 minutes, 21 to 22 minutes, 22 to 23 minutes, 23 to 24 minutes, 24 to 25 minutes, 25 to 26 minutes, 26 to 27 minutes, 27 to 28 minutes, 28 to 29 minutes, 29 to 20 minutes, inclusive of the endpoints.

In some embodiments, the cells are irradiated for approximately 8.5 to 10.3 minutes.

As used herein a “reference dose location” refers to a position that has a reproducible and documented relationship relative to the maximum or minimum absorbed-dose position.

Dose Uniformity Ratio (DUR) refers to ratio of the maximum to the minimum absorbed dose within the process load. The concept is also referred to as the max/min dose ratio. In some embodiments, the internal Dose Uniformity Ratio (DUR) is calculated to be 1.18, DUR=maximum dose/minimum dose=2.91/2.45=1.18. In some embodiments, the minimum internal dose (average of all three runs) was located at position 9B (2.45 kGy), which is located below the bottom layer of vials in the approximate geometric center of the shipper cooler. In some embodiments, the maximum internal dose (average of all three runs) is located at position 1T (2.91 kGy), which was located above the vials inside the middle layer of boxes, in the outer corner of the shipper.

In some embodiments, the minimum and maximum dose rates achieved were calculated as follows: the exposure time for all three irradiation runs during the study was 233 minutes. In some embodiments, to calculate the minimum exposure time needed to ensure the minimum dose is achieved during irradiation, the minimum exposure time=target dose/minimum dose rate for the day of irradiation. In some embodiments, to calculate the maximum exposure time needed to ensure the maximum dose is not exceeded during irradiation, the maximum exposure time=target dose/maximum dose rate for the day of irradiation. In some embodiments, to order to ensure the minimum required dose is achieved without exceeding the maximum required dose, the average exposure time is calculated as, (min exposure time+max exposure time)/2. In some embodiment, following irradiation the minimum delivered dose is determined as: minimum delivered dose=exposure time*minimum dose rate. In some embodiments, after irradiation the maximum delivered dose is determined as: maximum delivered dose=exposure time*maximum dose rate

In order to allow for the most accurate depiction of internal delivered dose and ensure the minimum dose to the product is achieved without exceeding the maximum established dose when using reference dosimetry, the dose adjustment ratios from the minimum position and maximum position to each reference dosimeter must be calculated. Of these dose adjustment ratios, the highest reference to minimum ratio and lowest reference to maximum ratio are chosen and used in subsequent calculations.

In some embodiments, reference positions FC (front center) and RC (rear center) are used. In some embodiments, the overall average dose at position FC is calculated and determined to be 3.04 kGy. In some embodiments, the overall average dose at position RC for all three runs was calculated and determined to be 3.03 kGy. In some embodiment, dose adjustment ratios from the reference position to the minimum internal delivered dose and from the reference position to the maximum internal delivered dose are calculated for each of the reference positions. The dose adjustment ratio from the FC position to the minimum internal delivered dose is calculated as Average FC Dose/Average Minimum Dose=3.04/2.45=1.239. The dose adjustment ratio from the FC position to the maximum internal delivered dose is calculated as Average FC Dose/Average Maximum Dose=3.04/2.91=1.046. The dose adjustment ratio from the RC position to the minimum internal delivered dose is calculated as Average RC Dose/Average Minimum Dose=3.03/2.45=1.235. The dose adjustment ratio from the RC position to the maximum internal delivered dose is calculated as Average RC Dose/Average Maximum Dose=3.03/2.91=1.042.

In some embodiments, in order to determine the dose range to deliver to the reference dosimeters, the minimum target dose to the reference dosimeter is determined by multiplying the required minimum internal dose determined by the highest of the two reference to minimum ratios, (e.g., Required minimum internal dose*1.239=minimum reference dose). In some embodiments, the maximum target dose to the reference dosimeter is determined by multiplying the required maximum internal dose determined. (e.g., Required maximum internal dose*1.042=maximum reference dose). If a reference dosimetry is used during routine production, then in order to determine the internal delivered dose from the reference dose, the minimum reference dose is divided by 1.239, (e.g., reference dose/1.239=minimum internal dose). The maximum internal reference dose is determined by dividing the maximum reference dose by 1.042 (e.g., reference dose/1.239=maximum internal dose).

### L. Processing Parameters

As used herein “simulated or surrogate material” refers to material with similar characteristics to the actual material being tested that can be used in lieu of actual product or actual product that will not be distributed to market. In some embodiments, vial configuration is arranged as 81 vials in 9 rows of 9 vials each, each containing 0.6 mL of Cryopreserved Cells. No partial vial boxes are to be included but are to be filled with 0.6 mL of surrogate product. The number of coolers to be irradiated (one for every 3 dewars) are entered as the number of cartons. The dose range required for the product are entered into the dose range field in kGy.

### M. Pre Cooling of Irradiation Cooler

In some embodiments, one plane of the irradiation cooler is marked “front” per protocol. Two cardboard separators are used and the cooler is cooled for at least 30 minutes. In some embodiments, two and half (2½) layer of dry ice is placed on the bottom of the irradiation cooler and covered with one prepared cardboard separator and replace lid. The time when the irradiation cooler lid is replaced, recorded, signed and dated.

### N. Transfer of Vial Boxes

In some embodiments, transfer must be completed within five (5) minutes. In some embodiments, transfer must start at least 30 minutes after addition of dry ice. In some embodiments, dewars are opened in numerical order as each one is needed. In some embodiments, all vial boxes are orientated with the labeling towards the “rear” of the irradiation cooler. Removal of the rack from dewars is in numerical order. The vial boxes in the irradiation cooler are placed on top of the cardboard separator. time of placement (hour and minutes) of the first vial box in irradiation cooler is recorded. The rack is replaced in the dewar and the dewar closed. A second prepared cardboard separator is placed on top of the vial boxes and covered with dry ice. The irradiation cooler is received into the ODMS-RT system. The requested minimum dose is entered as 0.00 kGy and 0.01 is entered as the requested maximum dose.

### O. Calculation of Exposure Time for Irradiation

In some embodiments, the date of irradiation of the cryopreserved cells are entered into the Dose Rate Chart. In some embodiments, the minimum exposure time is calculated as: Requested min dose (Gy)÷Min dose rate (Gy/minutes)=Exposure time (Minutes). In some embodiments, the maximum exposure time is calculated as: Requested max dose (Gy)÷Max dose rate (Gy/minutes)=Exposure time (Minutes). In some embodiments, the average exposure time is calculated as: (Minimum Exposure Time+Maximum Exposure Time)/2=Average Exposure Time. The exposure time must be entered into the process timer in minutes and seconds. In order to do so, the residual minutes (decimal) from the average exposure time must be converted into seconds as follows: any residual minutes (decimal place) from [15.11.6]×60 seconds/minute=Seconds. The irradiation cooler is irradiated bottom down so the arrows are pointing up and will not be reoriented during irradiation.

### P. Post Irradiation

In some embodiments, the transfer is completed within 5 minutes. The time the last vial box is transferred from the cooler into the dewar does not exceed two hours from time the first vial box is placed in the cooler. An “irradiated” sticker is folded in half around the handle of the rack in each dewar so the ends stick to each other. The two ends of the sticker are stapled together. The cooler is opened and the top layer of ice and top cardboard separator is removed. A first Technician opens dewar 3 and removes the rack. A second Technician removes the top layer of vial boxes one at a time and hand them to the first Technician. The time (hours and minutes) the first vial box is removed from the irradiation cooler is recorded. The technicians responsible for transferring the vial boxes into the dewar and recording the times will sign and date. The total elapsed time from the first vial box being placed in the cooler to completion of transfer of last vial box from cooler to dewar is recorded.

### Q. Illustrative Embodiments

In some embodiments, the cells express a modified and secretable heat shock protein (i.e., gp96-Ig). In some embodiments, the cells express a secretable heat shock protein (i.e., gp96-Ig), for example, Viagenpumantucel-L.

In some embodiments, the cells harbor an expression vector comprising a nucleotide sequence that encodes a secretable vaccine protein (i.e., gp96-Ig). In some embodiments, the cells harbor an expression vector comprising a nucleotide sequence that encodes a secretable vaccine protein (i.e., gp96-Ig), for example, Vesigenurtacel-L.

In some embodiments, the cells are formulated in a buffer containing a saline solution. In some embodiments, cells are irradiated and suspended in buffered saline solution containing 0.5% HSA. In some embodiments, the buffer contains 20 mM sodium phosphate buffer pH 7.5, 0.5M NaCl, 3 nM MgCl₂ at about 50° C. In some embodiments, the buffer contains 20 mM sodium phosphate buffer pH 7.5, 0.5M NaCl, 3 mM MgCl2 and 1 mM ADP in a volume of 100 microliters at 37° C.

In some embodiments, the cells are formulated in a cryopreservative medium. In some embodiments, the cells are formulated in a cryopreservative medium at a 1:1 dilution ratio. In some embodiments, the cryopreservation medium comprises, 20×10⁶ cell/mL, 0.5% HSA, 0.007% sodium bicarbonate, 0.567% sodium chloride, 5% DMSO and 6% Pentastarch. Cells are formulated fresh by a 1:1 dilution with cryopreservation medium to yield a final concentration of 20×10⁶ viable cells/mL containing 6% Pentastarch, 5% DMSO, 0.5% HSA, 0.007% sodium bicarbonate and 0.567% sodium chloride.

In some embodiments, the formulated cells are irradiated using Cobalt irradiator (Co⁶⁰). In some embodiments, the cells are irradiated at a dose of about 1 (Gy), 5 (Gy), 10 (Gy), 20 (Gy), 30 (Gy), 40 (Gy), 50 (Gy), 60 (Gy), 70 (Gy), 80 (Gy), 90 (Gy), 100 (Gy), 110 (Gy) or 120 (Gy) inclusive of the endpoints. In some embodiments, the dose rate is 120 (Gy). In some embodiments, aliquot and cryopreserved cancer vaccine cells in vials are irradiated with 120 (Gy) on dry ice. In some embodiments, the cells are irradiated for about 1 to 2 minutes, 2 to 3 minutes, 3 to 4 minutes, 4 to 5 minutes, 5 to 6 minutes, 6 to 7 minutes, 7 to 8 minutes, 8 to 9 minutes, 9 to 10 minutes inclusive of the endpoints. In some embodiments, the cells are irradiated for about 8.5 to 10.3 minutes.

In some embodiments, the crysopreservation medium comprises, 2×10⁶ cell/mL, 0.5% HSA, 0.007% sodium bicarbonate, 0.567% sodium chloride, 5% DMSO and 6% Pentastarch. Cells are diluted fresh in the wash medium (0.5% HSA, 0.007% sodium bicarbonate and 0.9% sodium chloride) to a concentration of 4×10⁶ cells/mL and immediately formulated by a 1:1 dilution ratio with cryopreservation medium. In some embodiments, the formulated cells are irradiated using Cobalt irradiator (Co⁶⁰).

In some embodiments, the cells are irradiated at a dose of about 20 (Gy), 25 (Gy), 30 (Gy), 35 (Gy), 40 (Gy), 45 (Gy), 50 (Gy), 55 (Gy), 60 (Gy), 65 (Gy), 70 (Gy), 75 (Gy), 80 (Gy), 85 (Gy), 90 (Gy), 95 (Gy), 100 (Gy), 110 (Gy), 115 (Gy), 120 (Gy), 125 (Gy), 130 (Gy), 135 (Gy), 140 (Gy), 145 (Gy), 150 (Gy), 155 (Gy), 160 (Gy), 165 (Gy), 170 (Gy), 175 (Gy), 180 (Gy), 185 (Gy), 190 (Gy), 195 (Gy), 200 (Gy), 210 (Gy), 215 (Gy), 220 (Gy), 225 (Gy), 230 (Gy), 235 (Gy), 240 (Gy), 245 (Gy), 250 (Gy), 255 (Gy), 260 (Gy), 265 (Gy), 270 (Gy), 275 (Gy), 280 (Gy), 285 (Gy), 290 (Gy), 295 (Gy), 300 (Gy), 320 (Gy), 325 (Gy), 330 (Gy), 35 (Gy), 340 (Gy), 350 (Gy), 360 (Gy), 365 (Gy), 370 (Gy), 375 (Gy), 380 (Gy), 385 (Gy), 390 (Gy), 400 (Gy), 425 (Gy), 430 (Gy), 435 (Gy), 440 (Gy), 445 (Gy), 450 (Gy), 460 (Gy), 465 (Gy), 470 (Gy), 475 (Gy), 480 (Gy), 485 (Gy), 490 (Gy), 495 (Gy), 500 (Gy), inclusive of the endpoints. In some embodiments, the dose rate is 120 (Gy). In some embodiments, aliquot and cryopreserved cancer vaccine cells in vials are irradiated with 120 (Gy) on dry ice. In some embodiments, the cells are irradiated for about 1 to 2 minutes, 2 to 3 minutes, 3 to 4 minutes, 4 to 5 minutes, 5 to 6 minutes, 6 to 7 minutes, 7 to 8 minutes, 8 to 9 minutes, 9 to 10 minutes, inclusive of the endpoints. In some embodiments, the cells are irradiated for about 8.5 to 10.3 minutes.

For use with HS-410, the cells are formulated in a cryopreservative medium at a 1:1 dilution ratio. In some embodiments, the cryopreservation medium comprises, 20×10⁶ cell/mL, 0.5% HSA, 0.007% sodium bicarbonate, 0.567% sodium chloride, 5% DMSO and 6% Pentastarch. Cells are formulated fresh by a 1:1 dilution with cryopreservation medium to yield a final concentration of 20×10⁶ viable cells/mL containing 6% Pentastarch, 5% DMSO, 0.5% HSA, 0.007% sodium bicarbonate and 0.567% sodium chloride. In some embodiments, the formulated cells are irradiated using Cobalt irradiator. In some embodiments, aliquot and cryopreserved cells in vials are irradiated with 120 (Gy) on dry ice. In some embodiments, the cells are irradiated for about 8.5 to 10.3 minutes.

For use with HS-110, the cells are formulated in a cryopreservative medium at a 1:1 dilution ratio. In some embodiments, the cryopreservation medium comprises, 20×10⁶ cell/mL, 0.5% HSA, 0.007% sodium bicarbonate, 0.567% sodium chloride, 5% DMSO and 6% Pentastarch. Cells are formulated fresh by a 1:1 dilution with cryopreservation medium to yield a final concentration of 20×10⁶ viable cells/mL containing 6% Pentastarch, 5% DMSO, 0.5% HSA, 0.007% sodium bicarbonate and 0.567% sodium chloride. In some embodiments, the formulated cells are irradiated using Cobalt irradiator. In some embodiments, aliquot and cryopreserved cells in vials are irradiated with 120 (Gy) on dry ice. In some embodiments, the cells are irradiated for about 8.5 to 10.3 minutes.

### 1. Assays of Cell Function

Surprisingly, freezing the cellular vaccine cells prior to irradiation does not generally change their characteristics, and provides significant benefits. These attributes are generally checked using one or more assays to determine cell viability, replication competency and metabolic function, as described below.

### a. Cell Viability Assays

In one embodiment, cell viability assays are done. In some embodiments, a CellTrace™ Violet Cell Proliferation Kit was used to access cell viability. CellTrace™ Violet stain crosses the plasma membrane and covalently binds inside cells where the fluorescent dye provides a consistent signal for several days in a cell culture environment. The dye binds covalently to all free amines on the surface and inside of cells and shows little cytotoxicity, with minimal observed effect on the proliferative ability or biology of cells. For cells that replicate and divide, the dye concentration in each cell is diluted with each division. Cells that do no grow do not show the same dilution of dye. Thus, the two populations can be distinguished on the basis of decreasing fluorescence as the membrane dye is diluted approximately equally between the dividing parental cell and the two resulting daughter cells.

In some embodiments, tritiated (³H)-thymidine incorporation methods are used to access cell viability. Thymidine incorporation assay, utilizes a strategy wherein a radioactive nucleoside, ³H-thymidine, is incorporated into new strands of chromosomal DNA during mitotic cell division. A scintillation beta-counter is used to measure the radioactivity in DNA recovered from the cells in order to determine the extent of cell division that has occurred in response to a test agent.

### b. Replication Competency Assays

In one embodiment, replication competency assays are done. As outlined herein, the cellular compositions for use as vaccines generally are replication incompetent, although they will remain viable for some time.

In some embodiments, a Clonogenic Assay (CFU) assay was used to confirm that the new irradiation process renders cells unable to replicate. In this CFU test, the culture substrate was the same type of monolayer cultures on tissue-treated polystyrene used for expansion of the cells in the manufacturing process. This CFU assay examined irradiated cells (and appropriate controls) for colonies of replicating cells after 21 days in culture.

### c. Metabolic Functionality Assays

In one embodiment, metabolic functionality assays are done. In some embodiments, the metabolic functionality assay is indicative of whether the cells in a culture are alive, by assessing metabolic rate; assessing relative contribution of aerobic (oxidative phosphorylation) versus anaerobic (glycolysis) processes for generation of ATP; measuring adherent cells in a microplate; or measure suspended cells in a microplate.

### EXAMPLES

In order that the invention disclosed herein may be more efficiently understood, examples are provided below. It should be understood that these examples are for illustrative purposes only and are not to be construed as limiting the invention in any manner.

### Example 1: Manufacturing Process and Process Controls

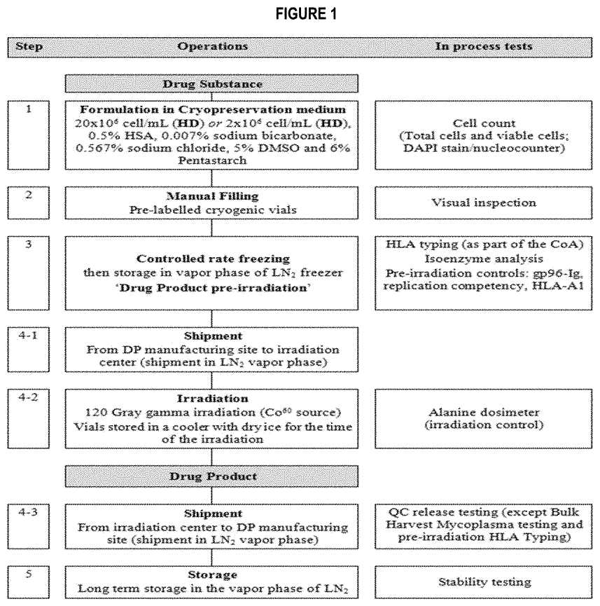

The manufacturing process for the Vesigenurtacel-L (HS-410) Drug Product consists of five Steps; Formulation, Vial fill, Freezing, Irradiation and Storage (see FIG. 1). The drug substance (bulk harvest of vesigenurtacel-L cells) is not stored but is immediately re-suspended in the final cryopreservation medium at the desired concentration and dispended into single-dose cryogenic vials to achieve the desired dose level. The vials are then frozen at a controlled rate and stored in the vapor of a liquid nitrogen freezer prior to irradiation. The irradiated vials constitute the final Drug Product. All open handling of the culture and expansion of the cells is conducted under sterile conditions in an ISO class 5 biosafety cabinet (BSC) within an ISO Class 7.

Formulation (Open System)

The Drug Substance (40×10⁶ cells/mL) is not stored, but immediately processed to generate the drug product. For the high strength formulation (High Dose), Drug Substance cells are formulated fresh by a 1:1 dilution with cryopreservation medium to yield a final drug concentration of 20×10⁶ viable cells/mL containing 6% Pentastarch, 5% DMSO, 0.5% HSA, 0.007% sodium bicarbonate and 0.567% sodium chloride.

For the low strength formulation (Low Dose), the Drug Substance is diluted fresh in the wash medium (0.5% HSA, 0.007% sodium bicarbonate and 0.9% sodium chloride) to a concentration of 4×10⁶ cells/mL and immediately formulated by a 1:1 dilution with cryopreservation medium to give a final drug concentration of 2×10⁶ viable cells/mL containing 6% Pentastarch, 5% DMSO, 0.5% HSA, 0.007% sodium bicarbonate and 0.567% sodium chloride.

CellTrace Violet Assay

To assess cell viability CellTrace™ Violet Cell Proliferation Kit was used. CellTrace™ Violet stain crosses the plasma membrane and covalently binds inside cells where the fluorescent dye provides a consistent signal for several days in a cell culture environment. The dye binds covalently to all free amines on the surface and inside of cells and shows little cytotoxicity, with minimal observed effect on the proliferative ability or biology of cells. For cells that replicate and divide, the dye concentration in each cell is diluted with each division. Cells that do no grow do not show the same dilution of dye. Thus the two populations can be distinguished on the basis of decreasing fluorescence as the membrane dye is diluted approximately equally between the dividing parental cell and the two resulting daughter cells.

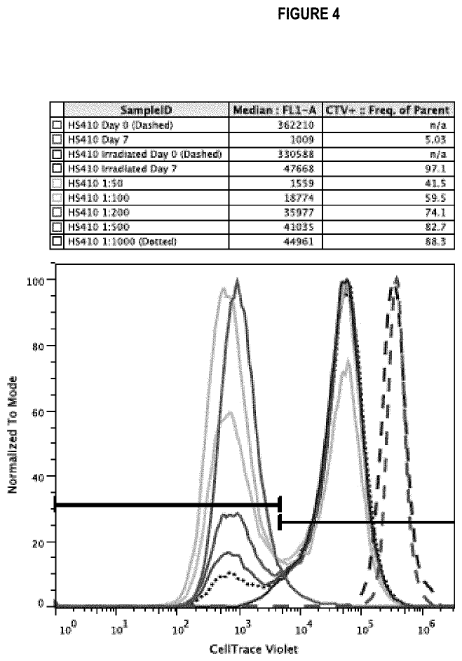

To assess the irradiation process as conducted in the Phase 2 manufacturing process, the CTV assay was used for assessment of the first GMP batches manufactured under this process (both High Dose and Low Dose batches, 140171149-HD and 140171149-LD). Per these Phase 2 process, these batches were irradiated in accordance with established Standard Operation Practice (SOPs). For the CTV assay, 20× High Dose HS-410 vials (12×10⁶ cells per 0.6 mL) and 10× Low Dose (1.2×10⁶ cells per 0.6 mL) were selected from different layers, boxes and vial locations (including lowest irradiated vials). The relative locations of vials tested for each box layer (T, M and B) and for both batches (High Dose and Low Dose) are shown in FIG. 4. Results show that for HS-410 cells, the CTV assay is sufficiently sensitive to detect 1 replication-competent cell in a background of 1000 non-replicating cells. For this cell line the CTV assay has a similar level of sensitivity to that observed with the tritiated (³H)-thymidine incorporation method (see FIG. 4).

Characterization of the Irradiation Process

Cryogenic vials from the first HS-410 GMP batches (High Dose and Low Dose) were tested for replication incompetence by two wholly distinct test methods: CellTrace Violet staining (CTV assay), and a clonogenic assay examining monolayer cultures on tissue-culture treated polystyrene (CFU assay). These assays were used rather than tritiated thymidine incorporation because the CTV assay and the CFU assay each specifically assesses cellular replication, whereas tritiated thymidine assessment detects DNA repair activities as well as actual replication. In the manufacturing of the HS-410 product, following the irradiation process, cells are expected to sustain DNA damage but to remain viable and metabolically active—thus it is expected that the cells may attempt DNA repair, but that ultimately the cells will be unable to replicate. Soft agar testing was considered as a possible test method to assess replication competence of these cells. However, testing of these cells with soft agar indicated that the HS-410 cell line is relatively adherence-dependent and does not grow well in soft agar. Therefore, soft agar is unlikely to be a sensitive assay method for detection of replication-competent cells in the HS-410 product.

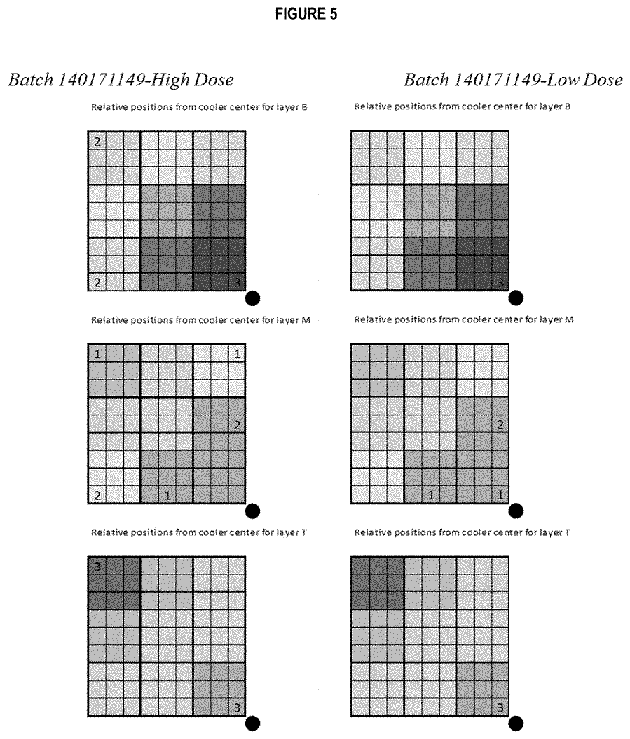

The CTV replication competence assay showed that the all vials from both batches tested (low-dose and high-dose batches) were replication-incompetent, in strong contrast to cells that were not exposed to irradiation. The data shown in FIG. 5 is representative of the CTV data obtained with cells from all the vials tested. The replication competence assay (CellTrace™ Violet (CTV) positive) met test control and validity criteria. In this assay, all irradiated cell cryovials tested demonstrated replication-incompetence by meeting the specification of >90% (min CTV+LD & HD=94.3%, Average CTV+HD=97.7%, Average CTV+LD=98.6%) of the CTV dye present in a non-replicating cell population compared to a control replicating HS-410 cell population after 7 days of culture. Additionally, cell counts (live and dead cells combined) were performed on Day 7, also demonstrating a lack of replication. 525,000 irradiated cells were plated on Day 0, and the average cell count on Day 7 was 487,816 and 507,430 cells for the HD and LD vials respectively, indicating a lack of cell growth. (for technical reasons, cells for this assay are cultured for 7 days total. It is not feasible to perform FACS detection of these cells after longer culture periods, as the irradiated cells enlarge to a size that renders FACS detection impractical.)

Clonogenic Assay (Monolayer Culture)

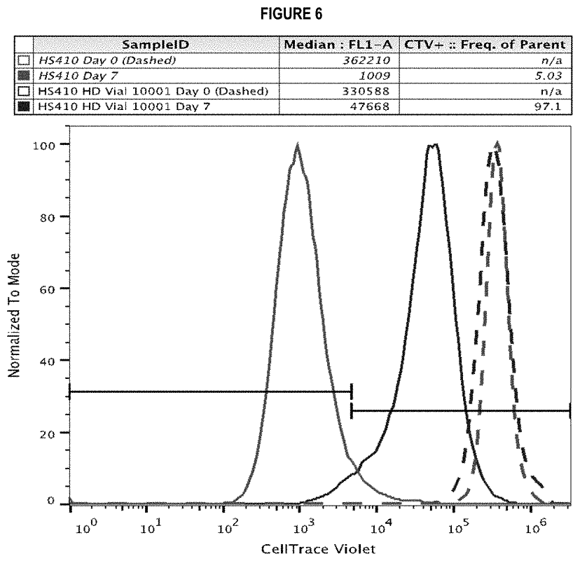

To provide additional support for the CTV assay, a second assay method was used to confirm that the new irradiation process renders HS-410 cells unable to replicate. In this CFU test, the culture substrate was the same type of monolayer cultures on tissue-treated polystyrene used for expansion of the cells in the manufacturing process. This CFU assay examined irradiated HS-410 cells (and appropriate controls) for colonies of replicating cells after 21 days in culture. The conditions of this assay were designed to conform to recommendations from FDA. For CFU testing of the initial GMP batch produced under the Phase 2 process, five High Dose HS-410 vials (12×10⁶ cells per 0.6 mL) and five Low Dose (1.2×10⁶ cells per 0.6 mL) were selected from five different boxes per batch, with four vials from each batch representing lowest-irradiated vials, and one vial per batch representing the location receiving the highest level of irradiation. The relative locations of vials tested for each box layer (T, M and B) and for both batches (High Dose and Low Dose) are shown in FIG. 6.

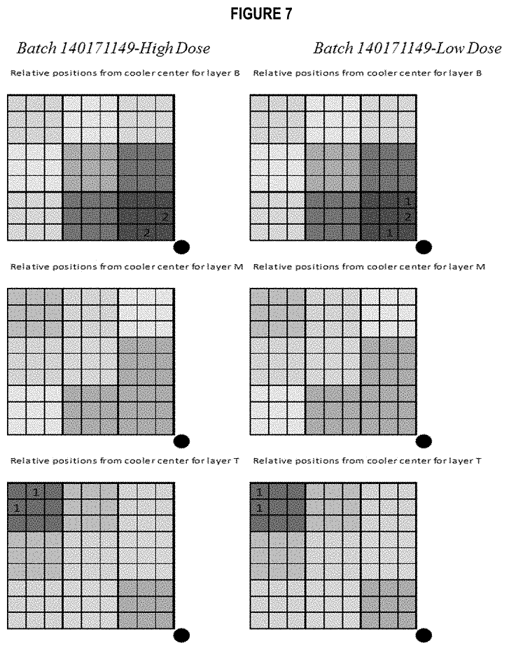

The CFU assay showed that the all vials from both batches tested (low-dose and high-dose batches) were replication-incompetent, in strong contrast to cells that were not exposed to irradiation. Controls for the assay (spiking a small number of non-irradiated cells into a much larger number of irradiated cells prior to plating the mixture) showed that at the seeding density used for these cultures, the assay sensitivity was at least 1/300,000 (the assay was sufficiently sensitive to detect one replication-competent cell in a background of 300,000+ irradiated cells which were unable to replicate). The CFU assay was conducted, plating the full contents of five product vials per batch (vials selected as shown above in FIG. 6 The same assay method was also utilized at an independent laboratory, examining smaller numbers of cells from this batch. Results from the independent laboratory also indicated that no replication-competent cells could be detected using this CFU method, (see FIG. 7).

### Example 2: Irradiation Process Validation

Irradiation Feasibility Study

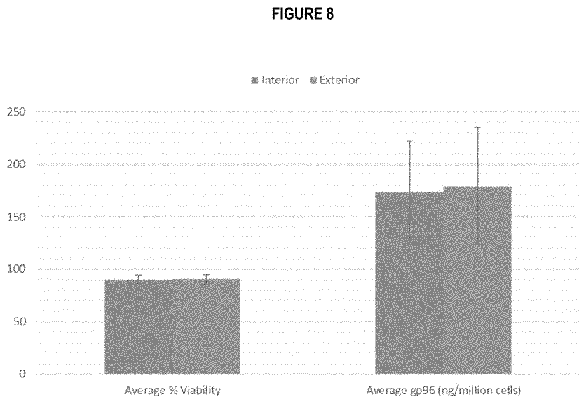

After having demonstrated that the shipping and handling procedures to irradiate cells at a different facility does not affect the viability nor the gp96-Ig expression, and that the target irradiation dose could be delivered to all areas of the Styrofoam cooler, a trial irradiation study was performed. Similar to the simulated irradiation study, each of 12 cryogenic boxes contained two frozen vials of HS-110 vaccine (12×10⁶ cells per 0.6 mL per vial) placed in exterior or interior areas of the cryogenic boxes. The rest of the vial slots of each cryogenic box contained frozen vials of cyropreservative medium. These cells were shipped and irradiated, following the established Standard Operation Practice (SOP). The irradiated vials were then shipped back to the manufacture in LN2 dewars and the cells tested for viability, HLA A1 and gp96-Ig expression, as well as replication competence. Each assay was performed on 3 vials of pre-irradiated and post-irradiated vials. In addition, vials containing cryopreservation media were tested for container closer integrity (dye immersion test). As shown in Table 3, below, no difference between the pre-irradiated and post-irradiated samples was observed for viability, recovery of viable cells, or HLA A1 or gp96-Ig expression. In addition, no difference was observed between cell vials that were expected to receive the maximum, minimum, or mid-irradiation dose, as determined by the Dose Mapping Study, (see FIG. 8).

TABLE 1
Viability, Recovery, and HLA A1 and
gp96-Ig Expression after Irradiation

| | | % Recovery | | |
|---|---|---|---|---|
| | % Vi- | of Viable | HLAA1 | gp96-Ig |
| Sample | ability⁽¹⁾ | cells⁽¹⁾ | (% positive)⁽¹⁾ | (ng/10⁶ cells)⁽¹⁾ |
| Pre- | 96.7 | 86.2 | 96.8 | 48 |
| irradiation (P) | | | | |
| Low Dose (L) | 96.2 | 91.8 | 96.6 | 41 |
| Medium Dose | 95.4 | 87.8 | 96.8 | 45 |
| High Dose (H) | 96.2 | 90.9 | 97.2 | 49 |

⁽¹⁾Average of 3 vials

Validation of Irradiation Process

To validate the irradiation process by demonstrating replication incompetence for the irradiated test article, cryogenic vials containing frozen HS-110 vaccine (12×10⁶ cells per 0.6 mL) in each of 12 cryogenic boxes was sent for irradiation in accordance with the established SOP. Forty vaccine vials were selected from the exterior and interior of each of 10 cryoboxes for testing in the replication competence assay. This number is a statistically appropriate number of samples to represent the entire batch, based on the sampling model in USP<71> for assessing sterility.

Briefly, Irradiated, non-irradiated and Mitomycin C (MMC) treated cells are thawed and placed into culture overnight in order to recover. The following day the cells are washed once in PBS and harvested by trypsinization. The cells are counted using a hemocytometer, and resuspended at 106 cells/mL in PBS. DMSO is added to a vial of CellTrace Violet to obtain a final concentration of 5 mM, and this is added to the cells to obtain a final concentration of 10 μM. The cells are incubated in the dark at 37 C for 20 min at which point unconjugated dye is quenched by adding 2-5 volumes of IMDM containing 10% FBS (CM1). 5×10⁶-1×10⁶ cells are removed, spun down, and resuspended in PBS for flow cytometric analysis. Irradiated and MMC treated cells were plated at 3×10³ cells/mL in a T175 flask containing 40 mL of CM1. Non-irradiated cells are plated at 3×10³ cells/mL in a T75 flask containing 25 mL of CM1. Cells are incubated at 37 C and 5% CO₂ for 7 days, then harvested by trypsinization and analyzed by flow cytometry. Gating is set such that ˜95% of the non-irradiated control cells on day 7 are in the CTV− population. Irradiated test samples were considered replication incompetent if they are >90% CTV+ on day 7.

Test articles were prepared by harvesting the cells 7 days after initial labeling. Spent medium was collected; cells were washed with PBS, and released from the flask by trypsinization. Trypsin is neutralized using the spent medium, and all flasks were washed once with PBS following neutralization. All washes were pooled with the spent medium and neutralized trypsin in order to harvest the greatest percentage of cells possible. Two controls are used in this assay. Non-irradiated HS-110 cells are used as a proliferating control and MMC treated HS-110 cells are used as a non-proliferating control. Assay were considered valid if all samples on day 0 show similar levels of labeling and there are cells available for harvest on day 7.

Evaluation of test results included comparing fluorescence levels at days 0 and 7 permits determining whether cells are undergoing active replication. Actively dividing cells will dilute the Celltrace Violet label much more efficiently than non-dividing cells, resulting in loss of fluorescence. 40 vials were tested in order to validate the irradiation process. 4 vials from each of 10 boxes were tested and labeled with the format box. Vial (e.g., 1.1, 1.2, . . . 10.4).

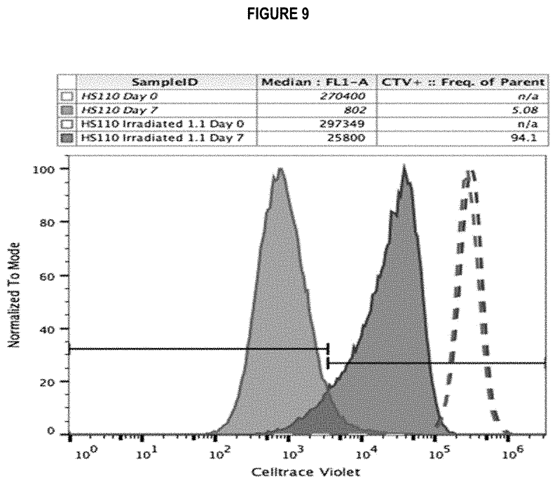

The replication competence assay showed that the all 40 vials tested were replication-incompetent, compared to cells that were not exposed to irradiation. The data shown in FIG. 9 is representative of the data obtained with cells from all 40 vials tested. The replication competence assay (CellTrace™ Violet (CTV) positive) met test control and validity criteria and all 40 irradiated cell cryovials tested demonstrated replication-incompetence by meeting the requirement of >90% of the CTV dye present in a non-replicating cell population compared to a control replicating HS-110 cell population after 7 days of culture. Additionally, cell counts (live and dead cells combined) were performed on Day 7, also demonstrating a lack of replication. For example, 525,000 irradiated cells were plated on Day 0, and the average cell count on Day 7 was 502,153 cells, indicating a lack of cell growth.

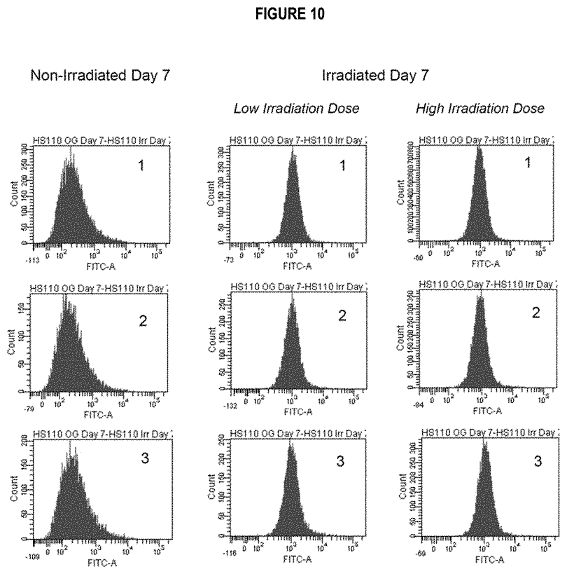

FIG. 10 shows the replication competency results for three vials of pre-irradiated cells and three vials of irradiated cells taken from minimum and maximum irradiation dose locations (based on the dose mapping data). These data suggest that all irradiated vials are replication-incompetent. All samples (vials with cryopreservation) tested for container closure integrity via the dye immersion test, including the pre-irradiated vial as well as those receiving low, medium, or high doses of irradiation, passed the test, indicating that the container closure system remained intact and functioned properly after irradiation.

All 40 irradiated vials met the requirement that >90% of cells be CTV+ on day 7. Table 2 outlines the results from every vial can be seen below. Samples that were read on the same day are shown in underline, italics, bold and bold+italics. Cell counts (live and dead cells combined) were performed on day 7, and they also demonstrated a lack of replication. 525,000 cells were plated on day 0, and the average cell count on day 7 was 502,153 cells.

| | Day 0 | Day 7 | Day 7 | Day 7 |
|---|---|---|---|---|
| Sample ID | MFI | MFI | CTV+ | Cell Count |
| HS110 #1 | 270400 | 802 | 5.08 | 8874000 |
| [symbol] | [symbol] | [symbol] | [symbol] | [symbol] |
| HS110 #3 | 308155 | 822 | 5.03 | 6656000 |
| HS110 #4 | 250427 | 610 | 5.03 | 5985000 |
| HS110 MMC #1 | 377065 | 101930 | 99.9 | 498270 |
| HS110 MMC #2 | 358664 | 110524 | 99.9 | 582000 |
| HS110 MMC #3 | 310677 | 110462 | 99.8 | 499550 |
| HS110 Irradiated 1.1 | 297349 | 25800 | 94.1 | 583000 |
| HS110 Irradiated 1.2 | 295184 | 24965 | 93.5 | 537000 |
| HS110 Irradiated 1.3 | 287730 | 23982 | 93.6 | 525300 |
| HS110 Irradiated 1.4 | 301727 | 24874 | 93.8 | 454140 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| HS110 Irradiated 2.4 | 285634 | 23206 | 93.4 | 47740 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| HS110 Irradiated 3.4 | 295184 | 24693 | 93.7 | 573000 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| HS110 Irradiated 4.4 | 288783 | 24603 | 93.6 | 550000 |
| [symbol] | [symbol] | [symbol] | [symbol] | [symbol] |
| HS110 Irradiated 5.2 | 298941 | 36930 | 95.8 | 249667 |
| HS110 Irradiated 5.3 | 294935 | 36190 | 95.5 | 549333 |
| [symbol] | [symbol] | [symbol] | [symbol] | [symbol] |
| HS110 Irradiated 6.1 | 281332 | 35108 | 95.2 | 519167 |
| HS110 Irradiated 6.2 | 285153 | 35705 | 95.7 | 503556 |
| [symbol] | [symbol] | [symbol] | [symbol] | [symbol] |
| [symbol] | [symbol] | [symbol] | [symbol] | [symbol] |
| HS110 Irradiated 7.1 | 293942 | 36558 | 95.3 | 485556 |
| HS110 Irradiated 7.2 | 262094 | 33040 | 94.8 | 473000 |
| HS110 Irradiated 7.3 | 280384 | 34990 | 95.1 | 546889 |
| HS110 Irradiated 7.4 | 255978 | 29752 | 96 | 512300 |
| HS110 Irradiated 8.1 | 298941 | 36806 | 95.4 | 481667 |
| HS110 Irradiated 8.2 | 306083 | 38978 | 95.8 | 473667 |
| HS110 Irradiated 8.3 | 291966 | 37813 | 96 | 480000 |
| HS110 Irradiated 8.4 | 234486 | 27154 | 95.9 | 585000 |
| HS110 Irradiated 9.1 | 244997 | 27655 | 96.4 | 390720 |
| HS110 Irradiated 9.2 | 245894 | 28580 | 96 | 534100 |
| HS110 Irradiated 9.3 | 241442 | 28895 | 96.2 | 377760 |
| HS110 Irradiated 9.4 | 242326 | 28789 | 96.3 | 452270 |
| HS110 Irradiated 10.1 | 264536 | 31199 | 96.1 | 407000 |
| HS110 Irradiated 10.2 | 239684 | 29535 | 96.5 | 510600 |
| HS110 Irradiated 10.3 | 248604 | 31313 | 96.8 | 515000 |
| HS110 Irradiated 10.4 | 298438 | 35715 | 97.2 | 500000 |

TABLE 3
Statistical data analysis: 100% of 40 tested irradiated
samples tested as replication incompetent.

| | Non-Irradiated | MMC | Irradiated |
|---|---|---|---|
| Statistic | HS110 | HS110 | HS110 |
| Avg. Day 0 MR | 268,271.25 | 348,802.00 | 275,580.90 |
| % CV Day 0 MFI | 9.31% | 8.02% | 8.07% |
| Avg. Day 7 MFI | 735.00 | 107,638.67 | 31,510.68 |
| % CV Day 7 MFI | 11.49% | 3.75% | 15.52% |
| Avg. Day 7 CTV+ | 5.04 | 99.87 | 95.39 |
| % CV Day 7 CTV+ | 0.43% | 0.05% | 1.06% |
| Avg. Day 7 Cell Count | 6,296,562.50 | 526,606.67 | 502,153.65 |
| % CV Day 7 Cell Count | 29.46% | 7.44% | 9.80% |

### Example 3: Comparative Study

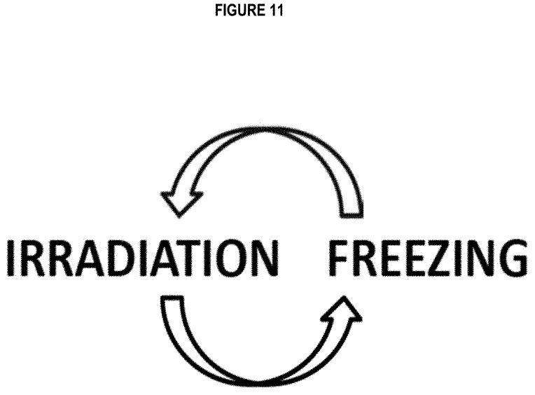

In prior irradiation procedure, the protocol included, a) Cell harvesting, b) Irradiation (12,000 Rad, in suspension, CM1 media, ice), c) Washing, cryopreservatives, vialing and d) Freezing to −70° C.→LN2 (see FIG. 11). Specifically, the cells were cultivated, harvested (in centrifugation tubes), resuspended in IMDM medium containing 9% FBS and irradiated as a bulk cell suspension (about 20×10⁶ cells per mL) in 250 mL centrifuge tubes on wet ice at dose of 120 Gray. After irradiation, the bulk vaccine was processed further by washing twice with wash medium and finally suspending the cells in the drug product cryopreservation medium before the aseptic filling and freezing step. Two bladder vaccine product batches manufactured were irradiated with this procedure. These batches (HBIB05 and HBIB06) were tested and shown to retain acceptable levels of cell viability and gp96-Ig expression post irradiation. In addition, the irradiated cells were shown to be replication-incompetent as assayed by FACs CellTrace™ Violet or tritiated (³H)-thymidine incorporation methods. Although results with this process were acceptable, to maintain cell viability the process required an irradiation facility in close proximity to (and tightly integrated with) the cell culture manufacturing facility. This combination is not common in the industry, and therefore was not feasible to retain after scaling up and transferring operations to a non-academic manufacturer.

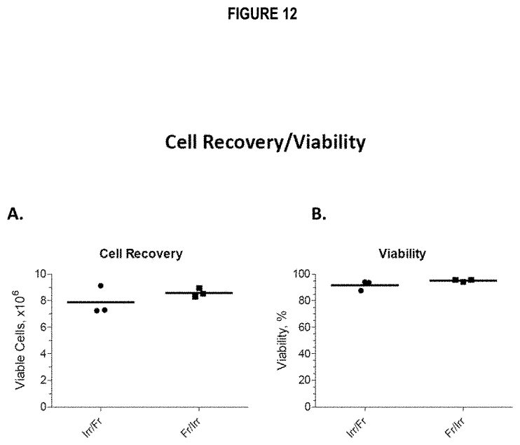

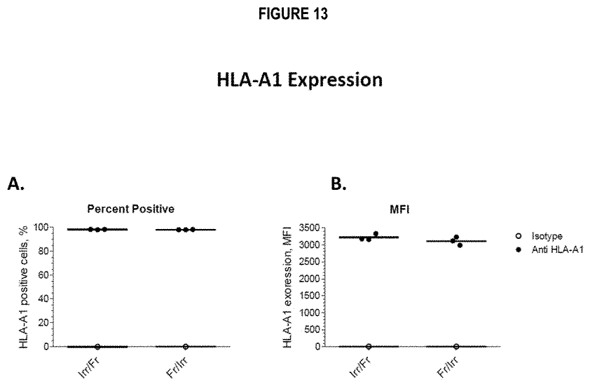

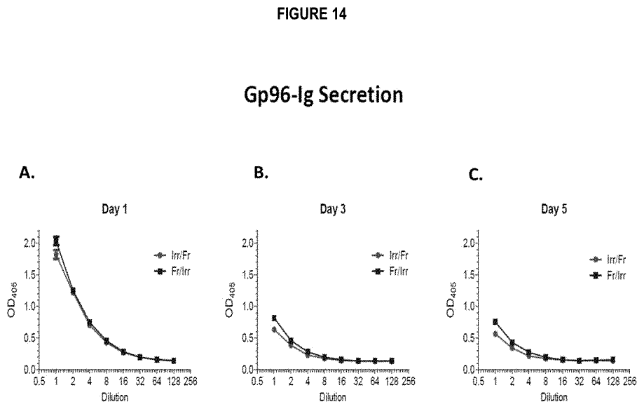

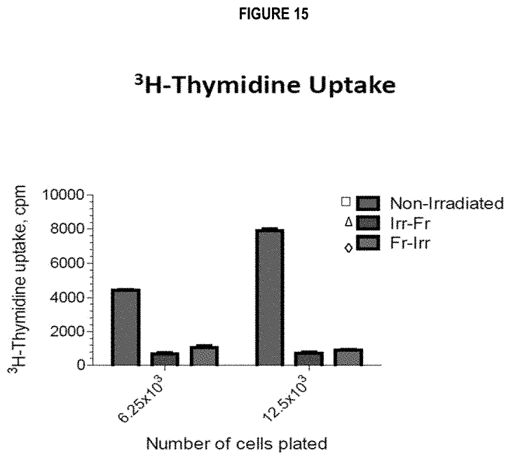

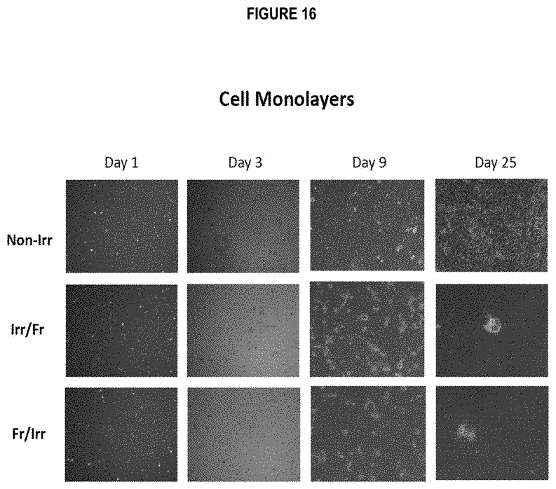

Thus, in the current methods of the present disclosure, the final product was formulated, filled into single-dose vials, and placed in cryogenic storage in a non-irradiated state. Only after the product was frozen was it then shipped to a separate facility for irradiation (frozen vials were shipped in LN2 dry shipper units, then transferred to a cooler packed with dry ice for the irradiation process). Procedure for the improved method includes, a) cells are harvested, b) washing, cryopreservatives, vialing, c) Freezing to −70° C., e) irradiation (12,000 Rad, vials on dry ice), and f) transfer to LN2. FIG. 12 and FIG. 13 compare the cell recovery, viability and HLA-A1 expression of Irradiated/Frozen (Irr/Fr) vs. Frozen/Irradiated cells (Fr/Irr). Results shows that cell viability, recovery and HLA-A1 expression is slightly improved following freezing and irradiation. Comparison of Elisa data of GP96-Ig secretion in irradiated/frozen (Irr/Fr) and frozen/irradiated cells (Fr/Irr), shows a significant increase in GP96-Ig following freezing and irradiation conditions (see FIG. 14). Comparison of thymidine uptake among non-irradiated, Irradiated/Frozen (Irr/Fr) and Frozen/Irradiated cells (Fr/Irr) cells also indicate an improvement following freezing and irradiation (see FIGS. 15 and 16).

Overall, the improved methods maintain cell viability does not require an irradiation facility in close proximity to (or to be tightly integrated with) the cell culture manufacturing facility, thereby making it feasible for scaling up and transfer.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any way.

1. A method for preserving cells, the method comprising:
a) obtaining freshly harvested cells in a container, wherein the cells express a modified and secretable vaccine protein;
b) contacting the harvested cells with liquid nitrogen; and
c) administering ionizing radiation (IR) to the cells at a dose of at least 120 (Gy).

2. The method of claim 1, further comprising storing the cells in liquid nitrogen.

3. The method of claim 1, wherein the method increases cell viability.

4. The method of claim 1, wherein the method increases cell recovery.

5. The method of claim 1, wherein the administration of the IR to the cells renders the cells replication incompetent.

6. The method of claim 1, wherein the cells are irradiated with gamma radiation.

7. The method of claim 6, wherein the cells are non-proliferative when administered gamma radiation.

8. The method of claim 1, wherein the dose of administered IR is selected from 120 (Gy), 125 (Gy), 130 (Gy), 135 (Gy), 140 (Gy), 145 (Gy), 150 (Gy), 155 (Gy), 160 (Gy), 165 (Gy), 170 (Gy), 175 (Gy), 180 (Gy), 185 (Gy), 190 (Gy), 195 (Gy), 200 (Gy), 210 (Gy), 215 (Gy), 220 (Gy), 225 (Gy), 230 (Gy), 235 (Gy), 240 (Gy), 245 (Gy), 250 (Gy), 255 (Gy), 260 (Gy), 265 (Gy), 270 (Gy), 275 (Gy), 280 (Gy), 285 (Gy), 290 (Gy), 295 (Gy), 300 (Gy), 320 (Gy), 325 (Gy), 330 (Gy), 335 (Gy), 340 (Gy), 350 (Gy), 360 (Gy), 365 (Gy), 370 (Gy), 375 (Gy), 380 (Gy), 385 (Gy), 390 (Gy), 400 (Gy), 425 (Gy), 430 (Gy), 435 (Gy), 440 (Gy), 445 (Gy), 450 (Gy), 460 (Gy), 465 (Gy), 470 (Gy), 475 (Gy), 480 (Gy), 485 (Gy), 490 (Gy), 495 (Gy), and 500 (Gy).

9. (canceled)

10. The method of claim 1, wherein the modified and secretable vaccine protein is a heat shock protein.

11. The method of claim 10, wherein the heat shock protein is gp96.

12. The method of claim 1, wherein the cells are tumor cells.

13. The method of claim 12, wherein the tumor cells are lung or bladder tumor cells.

14. The method of claim 12, wherein the tumor cells are viagenpumatucel-L (HS-110) cells.

15. The method of claim 12, wherein the tumor cells are vesigenurtacel-L (HS-410) cells.

16. (canceled)

17. The method of claim 1, further comprising expanding the cells in culture.

18. A method for making a cancer treatment, comprising:
a) obtaining freshly harvested cells in a container, wherein the cells are tumor cells comprising a vector encoding a modified and secretable vaccine protein;
b) contacting the harvested cells with liquid nitrogen; and
c) administering a dosage of ionizing radiation (IR) to the cells at a dose of at least 120 (Gy).

19. The method of claim 18, further comprising, storing the cells in liquid nitrogen.

20. The method of claim 18, wherein the modified and secretable vaccine protein is gp96-Ig.

21. The method of claim 18, wherein the cancer treatment is vesigenurtacel-L (HS-110) or vesigenurtacel-L (HS-410).
